(19) United States
(12) Patent Application Publication
(10) Pub. No.: US 2021/0330961 A1
(43) Pub. Date: Oct. 28, 2021

(54) IMPLANTABLE LVAD PUMP CONTROLLER HEADER

(71) Applicant: Medtronic, Inc., Minneapolis, MN (US)

(72) Inventors: Lisa Meyer, Faribault, MN (US); Andrew Ries, Lino Lakes, MN (US); Thomas J. Fick, Minneapolis, MN (US); Kevin Seifert, Forest Lake, MN (US); Richard P. Nelson, Hudson, WI (US); Jonathan A. Hughes, Blaine, MN (US)

(21) Appl. No.: 17/202,451

(22) Filed: Mar. 16, 2021

Related U.S. Application Data
(60) Provisional application No. 63/015,861, filed on Apr. 27, 2020.

(51) Int. Cl. A61M 60/878; A61N 1/375

(52) U.S. Cl. CPC A61M 60/878; A61N 1/37512; A61N 1/3752

(57) ABSTRACT

A connector block for an implantable controller. The connector block includes a first pair of side-by-side bores extending distally from a proximal end of the connector block and defining a major longitudinal axis. A second pair of side-by-side bores extends distally from a distal end of the first pair of side-by-side bores, each of the first pair of side-by-side bores defining a first diameter and each of the second pair of side-by-side bores defining a second diameter less than the first diameter, each pair of the second pair of side-by-side lead bores include a setscrew bore sized to receive a setscrew and being transverse to the major longitudinal axis.

### CROSS-REFERENCE TO RELATED APPLICATION

This application claims the benefit of U.S. Application Ser. No. 63/015,861, filed Apr. 27, 2020.

### FIELD

The present technology is generally related to connector blocks or implantable controllers.

### BACKGROUND

Controllers for implantable medical devices, such as pacemakers or ventricular assist devices, are configured to be coupled to one or more conductors that control operation of the implanted medical device. Because these controllers are implanted within the body, such conductors may be exposed to blood, or other body tissues, and thus there is a risk of corrosion or otherwise the possibility of an electrical short between the controller and the one or more conductors.

### SUMMARY

The techniques of this disclosure generally relate to connector assembly blocks for implantable medical devices.

In one aspect, the present disclosure provides a connector block for an implantable controller. The connector block includes a first pair of side-by-side bores extending distally from a proximal end of the connector block and defining a major longitudinal axis. A second pair of side-by-side bores extends distally from a distal end of the first pair of side-by-side bores, each of the first pair of side-by-side bores defining a first diameter and each of the second pair of side-by-side bores defining a second diameter less than the first diameter, each pair of the second pair of side-by-side lead bores include a setscrew bore sized to receive a setscrew and being transverse to the major longitudinal axis.

In another aspect of this embodiment, the connector block of claim 1, further comprising a third pair of side-by-side bores extending distally from a distal end of the second pair of side-by-side lead bores.

In another aspect of this embodiment the distal end of the second pair of side-by-side bores tapers in diameter to define a chamfered edge as it extends toward the third pair of side-by-side bores.

In another aspect of this embodiment, the first, second, and third pairs of side-by-side bores are coaxial.

In another aspect of this embodiment, the connector block further includes one or more spaced apart ribs disposed about the connector block.

In another aspect of this embodiment, the setscrew bore includes a setscrew stop recessed from an exterior surface of the connector block.

In another aspect of this embodiment, the stop is spaced a distance from the exterior surface of the connector block such that when the setscrew is inserted within the setscrew bore, a distal end of the setscrew is spaced a distance from an outer diameter of connector inserted within the second pair of side-by-side bores.

In another aspect of this embodiment, the connector block is sized to be received within an implantable controller.

In another aspect of this embodiment, the distal end of first pair of side-by-side bores defines a right angle with a proximal end of the second pair of side-by-side bores.

In another embodiment, a controller for an implantable blood pump includes a housing and a connector block disposed within a portion of the housing. The connector block is configured to receive a first connector in communication with the blood pump and a second connector from an implanted transcutaneous energy receiving coil, the connector block comprising. A first pair of side-by-side bores extends distally from a proximal end of the connector block and defining a major longitudinal axis. A second pair of side-by-side bores extends distally from a distal end of the first pair of side-by-side bores, each of the first pair of side-by-side bores defining a first diameter and each of the second pair of side-by-side bores defining a second diameter less than the first diameter, each pair of the second pair of side-by-side bores include a setscrew bore sized to receive a setscrew and being transverse to the major longitudinal axis.

In another aspect of this embodiment, the controller includes a third pair of side-by-side bores extending distally from a distal end of the second pair of side-by-side lead bores.

In another aspect of this embodiment, a distal end of the second pair of side-by-side bores tapers in diameter to define a chamfered edge as it extends toward the third pair of side-by-side bores.

In another aspect of this embodiment, the first, second, and third pairs of side-by-side bores are coaxial.

In another aspect of this embodiment, further including a plurality of spaced apart ribs circumferentially disposed about the connector block.

In another aspect of this embodiment, the setscrew bore includes a setscrew stop recessed from an exterior surface of the connector block.

In another aspect of this embodiment, the stop is spaced a distance from the exterior surface of the connector block such that when the setscrew is inserted within the setscrew bore, a distal end of the setscrew is spaced a distance from an outer diameter of connector inserted within the second pair of side-by-side bores.

In another aspect of this embodiment, the distal end of first pair of side-by-side bores defines a right angle with a proximal end of the second pair of side-by-side bores.

In another aspect of this embodiment, when the connector block is disposed within the housing it is substantially flush within an exterior surface of the controller.

In another aspect of this embodiment, when the setscrew is inserted within the setscrew bore, the setscrew is recessed from an exterior surface of the controller and electrically isolated from a respective one of the first connector and the second connector.

In another embodiment, a connector block for an implantable controller includes a first pair of side-by-side bores extending distally from a proximal end of the connector block and defining a major longitudinal axis. A second pair of side-by-side bores extends distally from a distal end of the first pair of side-by-side bores, each of the first pair of side-by-side bores defining a first diameter and each of the second pair of side-by-side bores defining a second diameter less than the first diameter, each pair of the second pair of side-by-side lead bores include a setscrew bore sized to receive a setscrew and being transverse to the major longitudinal axis, the setscrew bore includes a setscrew stop recessed from an exterior surface of the connector block, the stop is spaced a distance from the exterior surface of the connector block such that when the setscrew is inserted within the setscrew bore, a distal end of the setscrew is spaced a distance from an outer diameter of connector inserted within the second pair of side-by-side bores. A third pair of side-by-side bores extends distally from a distal end of the second pair of side-by-side lead bores, the first, second, and third pairs of side-by-side bores are coaxial, the distal end of the second pair of side-by-side bores tapers in diameter to define a chamfered edge as it extends toward the third pair of side-by-side bores. A plurality of spaced apart ribs is circumferentially disposed about the connector block.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a front inside view of a fully implantable blood pump system constructed in accordance with the principles of the present application;

FIG. 2 a front perspective view of a connector housing of the controller shown in FIG. 1 with a connector block retained within and a plurality of lead connectors disposed within the connector block;

FIG. 3 is a see-through view of the connector housing and connector block shown in FIG. 2 with the lead connectors removed and a zoomed in view of the connector block;

FIG. 3A is a zoomed in view of the connector block shown in FIG. 3;

FIG. 4 is a cross-sectional view of the connector block shown in FIG. 3;

FIG. 5 is a zoomed in view of the connector block shown in FIG. 4 assembled into the housing shown in FIG. 3 with a setscrew and O-ring retained within the setscrew bore;

FIG. 6 is a front perspective view of another connector block constructed in accordance with the principles of the present application; and

FIG. 7 is a side cross-sectional view of a connector block and a driveline connector housing constructed in accordance with the principles of the present application.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

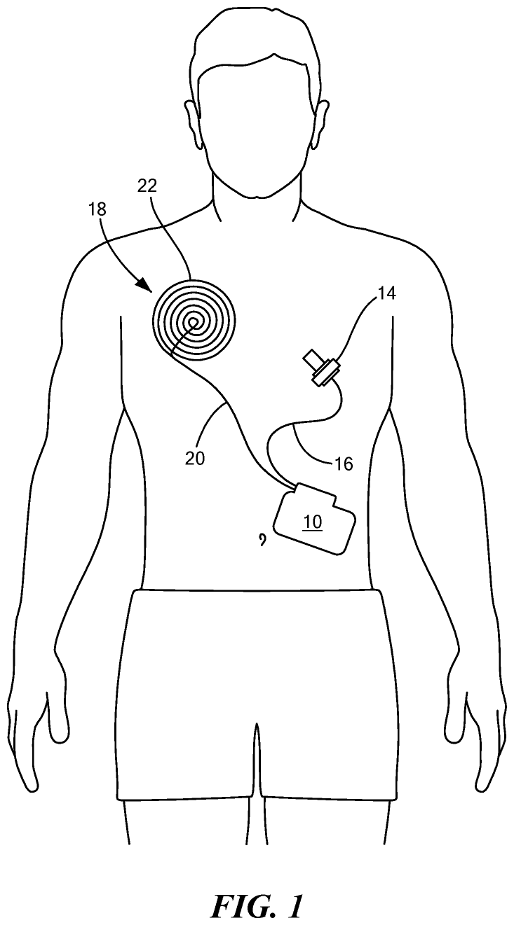

Referring to the drawings in which like reference designators refer to like elements, there is shown in FIG. 1 an exemplary implantable controller for an implantable medical device constructed in accordance with the principles of the present application and designated generally as “10.” The controller 10 may include one or more batteries 12 configured to power the components of the controller and provide power one or more implantable medical device, for example, a blood pump such as ventricular assist device (VAD) 14 implanted within the left ventricle of the patient's heart. VADs 14 may include centrifugal pumps, axial pumps, or other kinds electromagnetic pumps configured to pump blood from the heart to blood vessels to circulate around the body. One such centrifugal pump is the HVAD sold by HeartWare, Inc. and is shown and described in U.S. Pat. No. 7,997,854 the entirety of which is incorporated by reference. One such axial pump is the MVAD sold by HeartWare, Inc. and is shown and described in U.S. Pat. No. 8,419,609 the entirety of which is incorporated herein by reference. In an exemplary configuration, the VAD 14 is electrically coupled to the controller 10 by one or more implanted conductors that form a driveline 16 configured to provide power to the VAD 14, relay one or more measured feedback signals from the VAD 14, and/or provide operating instructions to the VAD 14. The controller 10 may include processing circuitry having one or more processors configured to operate the VAD 14 and to processes various signals received from the VAD 14.

Continuing to refer to FIG. 1, a receiving coil 18 may also be coupled to the controller 10 by, for example, one or more implanted conductors 20. In an exemplary configuration, the receiving coil 18 may be implanted subcutaneously proximate the thoracic cavity, although any subcutaneous position may be utilized for implanting the receiving coil 18. The receiving coil 18 is configured to be inductively powered through the patient's skin by a transmission coil (not shown) coupled to an external battery and controller (not shown) disposed opposite the receiving coil 18 on the outside of the patient's body. The receiving coil 18 may be disposed within a hermetically sealed package 22 that does not interfere with the conductivity of the receiving coil 18.

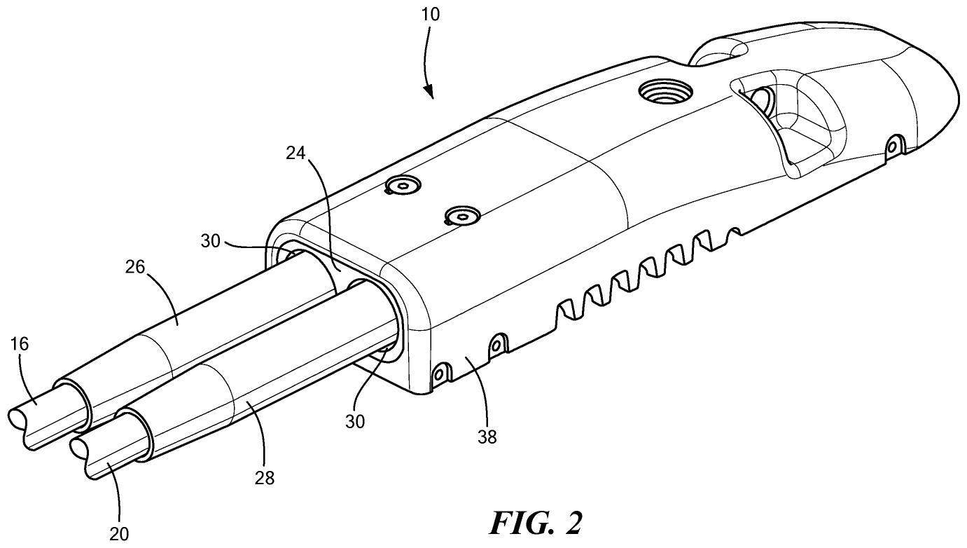

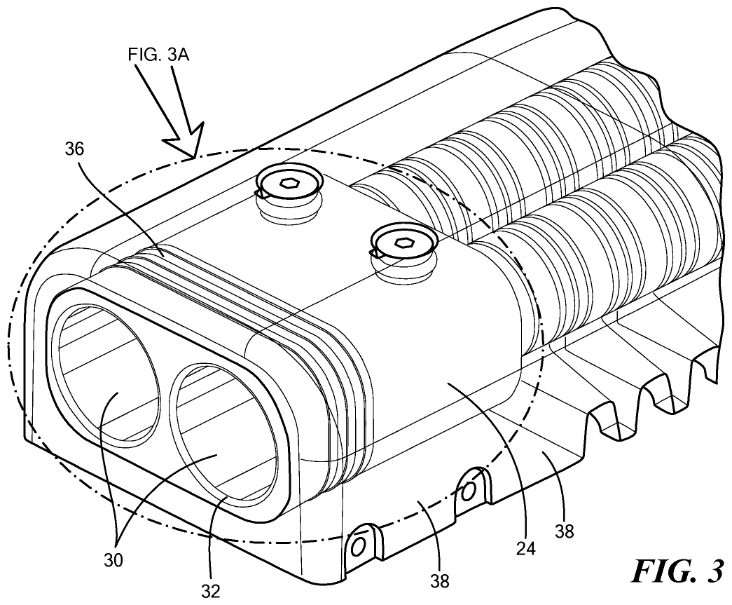

Referring now to FIGS. 2-4, the controller 10 may include a connector block 24 configured to receive a first connector 26 coupled to the distal end of the driveline 16, and a second connector 28 coupled to the distal end of the one or more implanted conductors 20. To that end, the connector block 24 includes a first pair of side-by-side bores 30 sized to receive the first connector 26 and the second connector 28. In the configuration shown in FIGS. 2-3, the first pair of side-by-side bores 30 are the same size, although in other configurations the size of each bore in the first pair of side-by-side bores 30 may be different depending on the size of the respective connector. As shown in FIG. 2, a proximal end 32 of the first pair of side-by-side bores 30, which is the entrance to the connector block 24, is substantially flush with an exterior surface of the controller 10. Each bore in the first pair of side-by-side bores 30 extends longitudinally and defines a respective major longitudinal axis extending from the proximal and 32 to a distal end 34 of the first pair of side-by-side bores 30. A plurality of ribs 36 (shown in FIG. 3 and FIG. 3A) may be disposed around the connector block 24 and proximate to the proximal end of the connector block 24 to aid in mechanical attachment of the connector block 24 within a housing 38 of the controller 10. In particular, the plurality of ribs 36 prevent the connector block 24 from moving within the housing 38 of the controller 10.

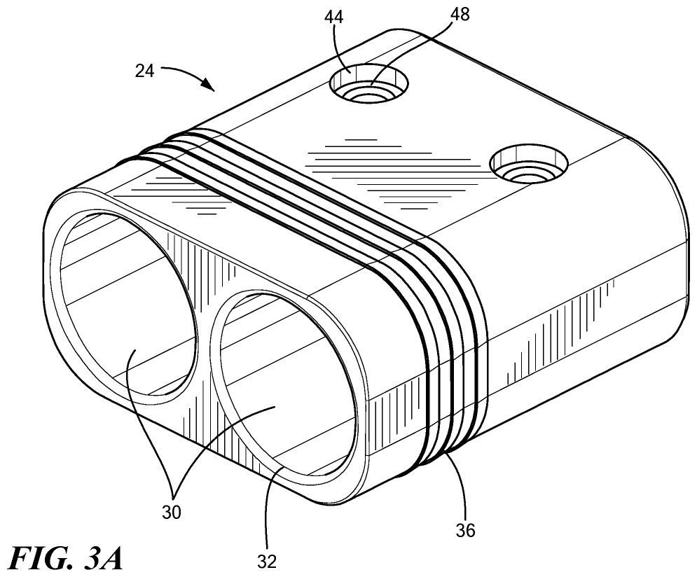

Referring now to FIG. 4, the connector block 24 includes a second pair of side-by-side bores 40 extending distally from a distal end of the first pair of side-by-side bores 30. Each of the first pair of side-by-side bores 30 defines a first diameter and each of the second pair of side-by-side bores 40 defines a second diameter less than the first diameter. The second pair of side-by-side bores 40 may be the same size or may be differently sized. In one configuration, the distal end of first pair of side-by-side bores 30 defines a right angle with a proximal end of the second pair of side-by-side bores 40. In other configurations, the junction between the first pair of side-by-side bores 30 and the second pair of side by-side bores 40 is an oblique angle, for example, a chamfered edge. Each bore of the second pair of side-by-side bores 40 extends along a respective major longitudinal axis extending from the distal end 34 of the first pair of side-by-side bores 30 to a distal end 42 of the second pair of side-by-side-bores 40. In one configuration, the first and second pairs of side-by-side bores are co-axial.

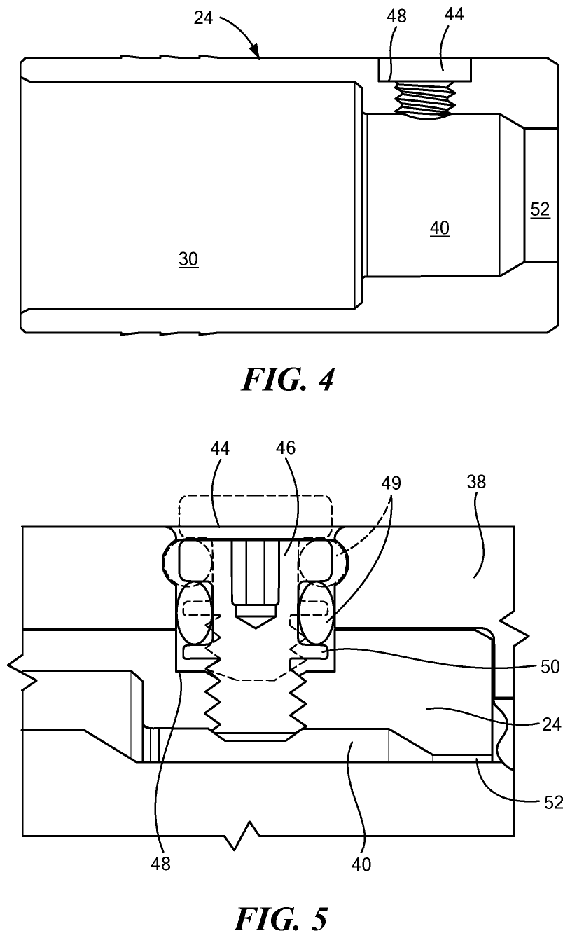

Continuing to refer to FIGS. 2-5, each bore of the second pair of side-by-side bores 40 define a setscrew bore 44 sized and configured to receive and retain a setscrew 46. Each setscrew bore 44 extends transverse to the major longitudinal axis, but in other configurations may be disposed, for example, at an oblique angle with respect to the major longitudinal axis. The setscrew 46 is configured to couple to the connector block 24 within the housing of the controller 10. In one configuration the setscrew bore 44 includes a stop 48 recessed from an exterior surface of the connector block 24. The stop 48 is configured to engage a corresponding flange 50 on the setscrew 46 such that the setscrew 46 is advanced to a predetermined position within the setscrew bore 44. For example, as shown in FIG. 5, the setscrew 46 is advanced within the setscrew bore 44 until the flange 50 contacts the stop 48. At this point, the setscrew 46 extends into the second pair of side-by-side bores 40, but does not contact any connector received within the bore such that the setscrew 46 is electrically isolated from the connector without the need for a grommet. Moreover, the setscrew 46 is recessed from the exterior surface of the connector block 24 when fully inserted within the setscrew bore 44. In the configuration shown in FIG. 5, an O-ring 49 is included around the setscrew 46 between a head of the setscrew 46 to provide a seal around the setscrew 46 and to prevent tissue ingrowth into the setscrew bore 44. In another configuration, the distal end of the setscrew bore 44 include a chamfered edge which causes the setscrew 44 to hit this chamfered edged before it contacts the stop 48 when inserted as an additional safety feature. Thus stop 48 prevents set screw 46 from contacting contacts on the connectors.

Additionally, there is a gap between the distal end of the setscrew 46 and the outer diameter of the connector within the second pair of side-by-side bores 40. This gap is created in part by the inclusion of a third pair of side-by-side bores 52 distal to and coaxial with the first and second pairs of side-by-side bores. As shown in FIG. 4, a proximal end of the third pair of side-by-side bores 52 defines an oblique angle with the distal end of the second pair of side-by-side bores 40. In one configuration, the distal end of the second pair of side-by-side bores 40 defines a chamfered edge with the third pair of side-by-side bores 52. That is, the distal end of the second pair of side-by-side bores 40 tapers in diameter toward the proximal end of the third pair of side-by-side bores 52. The third pair of side-by-side bores 52 defines a third diameter less than the second diameter and the first diameter.

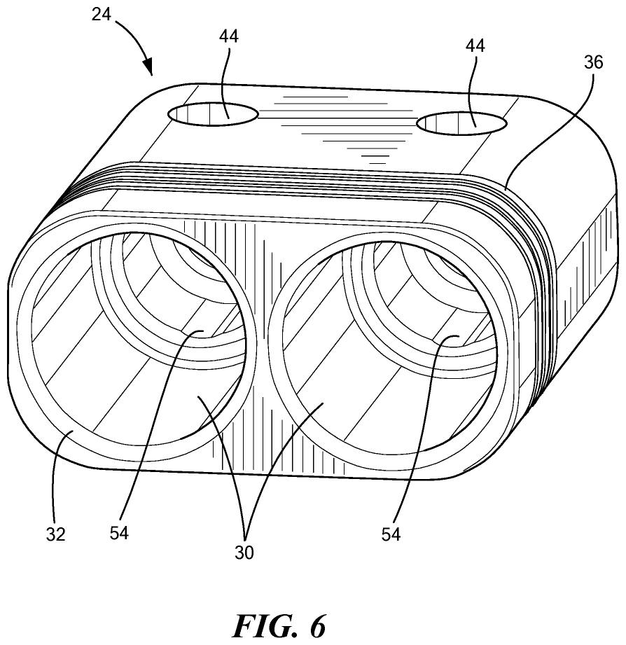

Referring now to FIG. 6, in another configuration, the second pair of side-by-side bores 40 or the first pair of side-by-side bores 30 may include a notch 54 or recess along a portion of their respective inner diameters. This notch 54 allows the first connector 26 and the second connector 28 to stabilized within their respective bore. For example, as shown in FIG. 5, the notch 54 is disposed opposite the setscrew bore 44 and the notch 54 is configured to provide stability of the connector within the connector block 24.

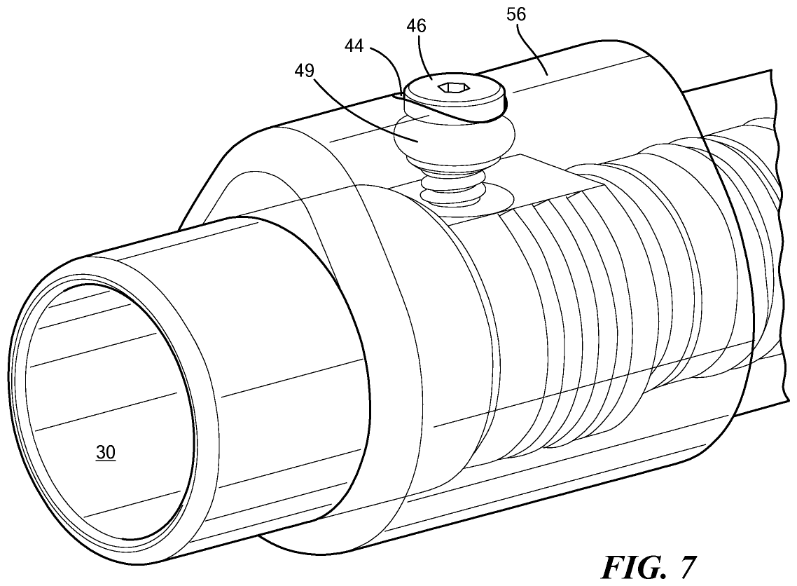

Referring now to FIG. 7, although the connector block 24 has been described having pairs of side-by-side bores, it is contemplated that only a single bore may be included as part of the design. For example, the connector block 24 may include a first bore 30 having a first diameter, a second bore 40 having a second diameter smaller than the first diameter, and a third bore 52 having a third diameter smaller than the second diameter, all arranged and configured in the same manner as described above. For example, the connector block having a single bore and including the features described above may be included in a driveline connector housing 56 as shown in FIG. 7. That is, first connector 26 may be inserted within connector block 24 or a connector block having a single bore for the driveline connector. Moreover, the setscrew 46 may be angled with respect to the connector block 24 rather than perpendicular to generate a forward bias to push the connector into position.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A connector block for an implantable controller, comprising:
a first pair of side-by-side bores extending distally from a proximal end of the connector block and defining a major longitudinal axis; and
a second pair of side-by-side bores extending distally from a distal end of the first pair of side-by-side bores, each of the first pair of side-by-side bores defining a first diameter and each of the second pair of side-by-side bores defining a second diameter less than the first diameter, each pair of the second pair of side-by-side lead bores include a setscrew bore sized to receive a setscrew and being transverse to the major longitudinal axis.

2. The connector block of claim 1, further comprising a third pair of side-by-side bores extending distally from a distal end of the second pair of side-by-side lead bores.

3. The connector block of claim 2, wherein the distal end of the second pair of side-by-side bores tapers in diameter to define a chamfered edge as it extends toward the third pair of side-by-side bores.

4. The connector block of claim 2, wherein the first, second, and third pairs of side-by-side bores are coaxial.

5. The connector block of claim 1, further including one or more spaced apart ribs disposed about the connector block.

6. The connector block of claim 1, wherein the setscrew bore includes a setscrew stop recessed from an exterior surface of the connector block.

7. The connector block of claim 6, wherein the stop is spaced a distance from the exterior surface of the connector block such that when the setscrew is inserted within the setscrew bore, a distal end of the setscrew is spaced a distance from an outer diameter of connector inserted within the second pair of side-by-side bores.

8. The connector block of claim 1, wherein the connector block is sized to be received within an implantable controller.

9. The connector block of claim 1, wherein the distal end of first pair of side-by-side bores defines a right angle with a proximal end of the second pair of side-by-side bores.

10. A controller for an implantable blood pump, comprising:
a housing;
a connector block disposed within a portion of the housing, the connector block being configured to receive a first connector in communication with the blood pump and a second connector from an implanted transcutaneous energy receiving coil, the connector block comprising:
a first pair of side-by-side bores extending distally from a proximal end of the connector block and defining a major longitudinal axis; and
a second pair of side-by-side bores extending distally from a distal end of the first pair of side-by-side bores, each of the first pair of side-by-side bores defining a first diameter and each of the second pair of side-by-side bores defining a second diameter less than the first diameter, each pair of the second pair of side-by-side bores include a setscrew bore sized to receive a setscrew and being transverse to the major longitudinal axis.

11. The controller of claim 10, further comprising a third pair of side-by-side bores extending distally from a distal end of the second pair of side-by-side lead bores.

12. The controller of claim 11, wherein a distal end of the second pair of side-by-side bores tapers in diameter to define a chamfered edge as it extends toward the third pair of side-by-side bores.

13. The controller of claim 12, wherein the first, second, and third pairs of side-by-side bores are coaxial.

14. The controller of claim 10, wherein the connector block further includes a plurality of spaced apart ribs circumferentially disposed about the connector block.

15. The controller of claim 10, wherein the setscrew bore includes a setscrew stop recessed from an exterior surface of the connector block.

16. The controller of claim 15, wherein the stop is spaced a distance from the exterior surface of the connector block such that when the setscrew is inserted within the setscrew bore, a distal end of the setscrew is spaced a distance from an outer diameter of connector inserted within the second pair of side-by-side bores.

17. The controller of claim 10, wherein the distal end of first pair of side-by-side bores defines a right angle with a proximal end of the second pair of side-by-side bores.

18. The controller of claim 10, wherein when the connector block is disposed within the housing it is substantially flush within an exterior surface of the controller.

19. The controller of claim 10, wherein when the setscrew is inserted within the setscrew bore, the setscrew is recessed from an exterior surface of the controller and electrically isolated from a respective one of the first connector and the second connector.

20. A connector block for an implantable controller, comprising:
a first pair of side-by-side bores extending distally from a proximal end of the connector block and defining a major longitudinal axis;
a second pair of side-by-side bores extending distally from a distal end of the first pair of side-by-side bores, each of the first pair of side-by-side bores defining a first diameter and each of the second pair of side-by-side bores defining a second diameter less than the first diameter, each pair of the second pair of side-by-side lead bores include a setscrew bore sized to receive a setscrew and being transverse to the major longitudinal axis, the setscrew bore includes a setscrew stop recessed from an exterior surface of the connector block, the stop is spaced a distance from the exterior surface of the connector block such that when the setscrew is inserted within the setscrew bore, a distal end of the setscrew is spaced a distance from an outer diameter of connector inserted within the second pair of side-by-side bores;
a third pair of side-by-side bores extending distally from a distal end of the second pair of side-by-side lead bores, the first, second, and third pairs of side-by-side bores are coaxial, the distal end of the second pair of side-by-side bores tapers in diameter to define a chamfered edge as it extends toward the third pair of side-by-side bores; and
a plurality of spaced apart ribs circumferentially disposed about the connector block.
